(19) United States
(12) Patent Application Publication
(10) Pub. No.: US 2010/0003296 A1
(43) Pub. Date: Jan. 7, 2010

(54) MANUFACTURING METHODS AND APPLICATIONS OF ANTIMICROBIAL PLANT FIBERS HAVING SILVER PARTICLES

(76) Inventors: Jiachong Cheng, Beijing (CN); Jixiong Yan, Wuhan (CN)

Correspondence Address: EVAN LAW GROUP LLC, 600 WEST JACKSON BLVD., SUITE 625, CHICAGO, IL, 60661, US

(21) Appl. No.: 11/722,412

(22) PCT Filed: Dec. 14, 2005
(86) PCT No.: PCT/CN2005/002182

§ 371 Date: Apr. 3, 2008

Related U.S. Application Data
(60) Provisional application No. 60/637,442, filed on Dec. 21, 2004.

(51) Int. Cl. A61K 9/70; A61K 33/38; A61P 31/04; A61P 31/10

(52) U.S. Cl. 424/404; 424/443; 424/618

(57) ABSTRACT

The present invention also provides a method for making the antimicrobial plant fibers. The characteristic of the method is no need of additional reducing agent. The present invention provides plant fibers with antimicrobial effects. The antimicrobial antifungal effect of the fibers is derived from nanosilver particles (diameter between 1 and 100 nm) which are attached to the fibers. The fibers which are made of cotton, linen, blending fibers, or any combination thereof. The fibers can be used to make yarn cloth to be used particularly for treating patients with burns or wound. The cloth made from the antimicrobial fibers can be further used to make clothes such as underwears, socks, shoe cushions, shoe linings, bed sheets, pillow cases, towels, women hygiene products, laboratory coats, and medical robes.

### FIELD OF THE INVENTION

The present invention relates to methods for making and using the antimicrobial fiber for healthcare and medical use. The present invention relates to antimicrobial fiber which is made from plant fiber and contains, preferably, about 0.1% to 1.5% by weight of nanosilver particles (diameter between 1 nm and 100 nm) attached thereto. A nanosilver content outside the aforementioned range may also provide satisfactory results. The nanosilver particles are prepared without the use of additional reducing agents. The antimicrobial fiber is preferably used in making cloth particularly for treatment of patients with burns or wounds. The cloth can be used to make clothes such as underwear, socks, shoe cushions, shoe linings, bed sheets, pillow cases, towels, women hygiene products, laboratory coats, and medical robes.

### DESCRIPTION OF THE RELATED ART

Metals including silver, copper, mercury, and zinc are known for anti-bacterial properties. Bacteria treated by these metals do not acquire resistance to the metals. Therefore, the bactericidal metals have advantages over the conventional antibiotics which often cause the selection of antibiotic-resistant microorganism.

Silver is generally a safe and effective antimicrobial metal. Silver ions function in adversely affecting cellular metabolism to inhibit bacterial cell growth. When silver ions are absorbed into bacterial cells, silver ions suppress respiration, basal metabolism of the electron transfer system, and transport of substrate in the microbial cell membrane. Silver has been studied for antibacterial purposes in the form of powder, metal-substituted zeolite, metal-plated non-woven fabric, and silver-containing crosslinked compound.

Nano technology is the study and treatment of substance and material in a nanometer range. Nanometer equals to 10⁻⁹ meter. The internationally acclaimed range for research and study for the nano technology is between 0.1 nm and 100 nm. The technology has been applied in the areas of information technology, energy, environment, and biotechnology. Particularly, the technology has been used in medicine including drug carrier, cell dye, cell separation, clinical diagnosis, and disinfection.

In the late eighteenth century, western scientists confirmed that colloidal silver, which had been used in oriental medicine for centuries, was an effective antibacterial agent. Scientists also knew that the human body fluid is colloidal. Therefore, colloidal silver had been used for antibacterial purposes in the human body. By the early nineteenth century, colloidal silver was considered the best antibacterial agent. However, after the discovery of antibiotics, due to the fact that antibiotics were more potent which could in turn generate more revenue, antibiotics had substituted colloidal silver as the main choice for antibacterial agents.

Thirty years after the discovery of the antibiotics, many bacteria developed resistance to the antibiotics, which became a serious problem. Since 1930s, silver, particularly colloidal silver, has once again been recognized for antibacterial use, particularly due to its ability for not causing drug-resistance.

Antibacterial cloth containing metallic particles (particularly copper, silver, and zinc in the form of zeolite) is known in the field for a long time. Many methods for incorporating the metal ions directly into a cloth or fabric have been proposed. However, in the methods in which the metals are used directly, the incorporation of metals lead to very expensive products, with heavy weights as they are necessarily used in a large amounts.

There are also methods teaching the use of a polymeric substance to hold the metallic ions. For example, the method of binding or adding fine wires or powder of the metals themselvers to a polymer and the methods of incorporating compounds of the metals into a polymer. However, the products obtained by these methods shows poor durability of antibacterial performance and can be utilized only for restricted purposes because the metal ions are merely contained in or attached to the polymer and, accordingly, they easily fall away from the polymer whiles being used.

For example, Japanese Patent No. 3-136649 discloses an antibacterial cloth used for washing breasts of milk cow. The Ag⁺ ions in AgNO₃ are crosslinked with polyacrylonitrile. The antibacterial cloth has demonstrated anti-bacterial activity on six (6) bacterial strains including Streptococcus and Staphylococcus.

Japanese Patent No. 54-151669 discloses a fiber treated with a solution containing a compound of copper and silver. The solution is evenly distributed on the fiber. The fiber is used as an anti-bacterial lining inside boots, shoes, and pants.

U.S. Pat. No. 4,525,410 discloses a mixed fiber assembly composed of low-melting thermoplastic synthetic fibers and ordinary fibers which are packed and retained with specific zeolite particles having a bactericidal metal ion.

U.S. Pat. No. 5,180,402 discloses a dyed synthetic fiber containing a silver-substituted zeolite and a substantially water-insoluble copper compound. The dyed synthetic fiber is prepared by incorporating a silver-substituted zeolite in a monomer or a polymerization mixture before the completion of polymerization in the step of preparing a polymer for the fiber.

U.S. Pat. Nos. 5,496,860 and 5,561,167 disclose antibacterial fiber including an ion exchange fiber and an antibacterial metal ion entrapped within the ion exchange fiber through an ion exchange reaction. The ion exchange fiber has sulfonic or carboxyl group as the ion exchange group.

U.S. Pat. No. 5,897,673 discloses fine metallic particles-containing fibers with various fine metallic particles therein, which have fiber properties to such degree that they can be processed and worked, and which can exhibit various functions of the fine metallic particles, such as antibacterial deodorizing and electron-conductive properties as provided.

U.S. Pat. No. 5,985,301 discloses a production process of cellulose fiber characterized in that tertiary amine N-oxide is used as a solvent for pulp, and a silver-based antibacterial agent and optionally magnetized mineral ore powder are added, followed by solvent-spinning.

The materials of the prior art involving the use of zeolite do not have sufficiently antibacterial activity due to lack of sufficient surface contact between the bactericidal metal and the bacteria, especially in water. The bactericidal activity of these materials rapidly diminishes as the silver ions become separated from the supports, especially in water. Most importantly, these materials do not show bactericidal activity over a prolonged period of time and the crosslinking may introduce compounds that cause allergy in patients.

There is yet another approach of making antibacterial cloth such as by inserting a layer of metallic yarn between a woven fabric. For example, Japanese laid-open patent publication (unexamined) No. Hei 6-297629 discloses an antibacterial cloth in which an inner layer member containing copper ion in a urethane foam resin is inserted in a cloth-like outer layer member. The outer layer member is composed of a cotton yarn serving as a weft formed by entangling an extra fine metallic yarn of copy or the like and a rayon yarn serving as a warp. A warp is the thread of a woven fabric which are extended lengthwise in the loom. A weft is the thread of a woven fabric that cross from side to side of the web and interlace the warp. This type of antibacterial cloth is heavy and hard. In addition, the extra fine metallic yarn is easy to cut, thus, causing problems to wash the cloth repeatedly. It may also injure a user due to the cut metallic yarn.

Recently, Chinese Patent No. 921092881 discloses a method for making antibacterial fabric with long lasting broad-spectrum antibacterial effect against more than 40 bacteria. The fabric is manufactured by dissolving silver nitrate in water, adding ammonia water into the solution to form silver-ammonia complex ion, adding glucose to form a treating agent, adding fabric into the treating agent, and ironing the fabric by electric iron or heat-rolling machine.

The present invention provides an antimicrobial fibers having nanosilver particles adhered thereto that is very effective over a broad spectrum of bacteria, fungi, and virus. The antimicrobial fiber of the present invention does not lose the antimicrobial strength over time, and the fiber is especially effective in water. The preferred fibers used in the present invention are entirely or at least partially plant fibers. Other types of fibers which are derivate of glucose may also be used to provide satisfactory results; their color can be natural or dyed. The antimicrobial fibers of the present invention is non-toxic, safe and thus suitable for use in healthcare related purposes.

The present invention also provides a method for making the antimicrobial fibers which is very simple, fast and easy to carry out. The use of reducing agents is completely eliminated in the process of the present invention, thus, the silver-containing processing solution is more stable and can be stored for much longer without precipitation of silver particle. The method of the present invention also produces reliable results and can be applied in small and industrial scale production.

### SUMMARY OF THE INVENTION

The present invention provides an antimicrobial fibers which contains nanosilver particles in the diameter of about 1-100 nm. The total weight of silver in the fibers is preferably about 0.1%-1.5% by weight. The nanosilver particles are attached to the fibers. Cotton, linen, blending fabric, or any combination therewith can be used as materials for the fibers. The fibers can be in its natural color or dyed with different colors.

The silver of the nanosilver particles is made by reducing silver ion or silver-ammonia complex without using additional reducing agent.

The fibers has antimicrobial effects against bacteria, fungi, and/or chlamydia, which include, but are not limited to, Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C.

The antimicrobial fibers can be used to make cloth (such as bandage, gauze, and surgical cloth) with antimicrobial activity, particularly to be used for treating patient with burn and scald-related skin infection, wound-related skin infection, dermal or mucosal bacterial or fungal infection, surgery cut infection, vaginitis, and acne-related infection.

Additionally, the cloth with antimicrobial activity can be used make antibacterial clothes or clothing such as underwear, socks, shoe cushions, shoe linings, bed sheets, pillow shams, towels, women hygiene products, laboratory coat, and patient clothes.

The present invention also provides methods for manufacturing the antimicrobial fibers. The method includes the following steps: (1) preparing a silver-containing solution with silver nitrate or other suitable silver salts with appropriate solubility in water, which dissociate to silver ion (Ag⁺), or with other silver salts without appropriate solubility in water and ammonia water, which form silver ammonia complex ion with improved, needed solubility in water. (2) soaking the plant fiber in the silver-containing solution or spraying the silver-containing solution to the plant fiber. (3) dehydrating or drying the plant fiber having absorbed silver-containing solution to form the antimicrobial fiber attached by silver particles with the size of 1-100 nm. Preferably, the plant fiber is predegreased before soaking in the silver-containing solution. After soaking in the silver-containing solution, the plant fiber may be treated with heat, for example, at 120° C.-200° C. for about 40-60 minutes. Other temperatures and duration may also provide satisfactory results.

For each liter of the silver containing solution, it is preferred that it contains 1 g-15 g of silver. The resulting nanosilver particles are sized between 1 nm to 100 nm in diameter and the antimicrobial fibers containing about 0.1% to 1.5% by weight of silver in a form of attached nanosilver particles.

### DETAILED DESCRIPTION OF THE INVENTION

The present invention provides methods to manufacture plant fiber which has a long-lasting effect and can be in the form of raw material, yarn, used in weaving and knitting to form cloth, or nonwoven cloth, composed of either natural or man-made fibers, or blend with synthetic fibers. The antimicrobial fibers contains nanosilver particles having diameters in the range of 1 nm to 100 nm. The nanosilver particles are attached to the fibers and contribute to the antimicrobial effects. The silver content in the antimicrobial fiber is 0.1% to 1.5% by weight of the total weight of the fibers.

The plant fibers are cotton, linen or blending fabric with synthetic fiber, or a combination therewith. The fibers can be either in its natural color or dyed with various colors, and the antimicrobial capacity of the fiber (either in natural color or dyed with various colors) is retained.

The antimicrobial fibers of the present invention is non-toxic, safe, and thus, suitable for use in medical or healthcare related purposes. The antimicrobial fibers can be used to make an antimicrobial yarn, cloth and nonwoven cloth. The cloth and nonwoven cloth are suitable for use as bandage, gauze or surgery cloth. They can also be used in making clothes or clothing such as underwear, panty, shoe cushions, shoe insole, shoe lining, bedding sheets, pillow sham, towel, feminine hygiene products, medical robes etc.

The term “antimicrobial” as used in the context of “antimicrobial fiber”, “antimicrobial yarn”, “antimicrobial cloth”, “antimicrobial nonwoven cloth”, and/or “antimicrobial clothes or clothing” in the present invention means that the fiber, yarn cloth, nonwoven cloth, or clothes (or clothing) has demonstrated antibacterial, antifingal, and anti-chlamydia effects by killing and/or suppressing growth of a broad spectrum of fungi, bacteria, and chlamydia, such as Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C.

The antimicrobial effect of the present invention is derived from silver ions which have advantage over the conventional antibiotics, as it does not induce resistance in the microorganisms. The antimicrobial fibers of the present invention does not lose the antimicrobial strength over time, and the antimicrobial effects are especially stronger in water.

Specially, the antimicrobial fibers of the present invention is suitable for use as cloth or clothes in disinfecting and treating patient with burn and scald-related skin infection, wound-related skin infection, skin or mucosa bacterial or fungal infection, surgery cut infection, vaginitis, and acne-related infection.

The well-known Silver-Mirror Reaction uses the reaction of silver nitrate aqueous solution with ammonia water to form silver ammonia complex ion, then the ion is reduced by glucose to form metallic silver.

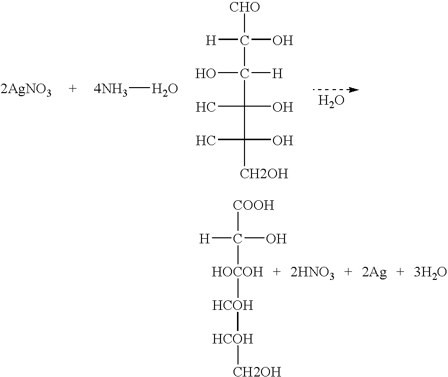

The existence of glucose reducing agent makes the mixed solution quick to react forming silver precipitate even at room temperature and the process difficult to control.

Some organic substances such as sugar and starch, can react with silver nitrate to form tiny silver particles. Sugar and starch are derivatives of glucose. The cellulose of the plant fibers is derivative of glucose too. As a particular example of the present invention, we found plant fiber can make silver nitrate solution (Ag⁺) or silver-ammonia complex ion solution [Ag(NH₃)₂⁺] reduce to form tiny silver particles at 120° C.-200° C. The silver-containing solution without reducing agents is stable and can be stored at room temperature for much longer time without forming silver particles, so the said silver-containing solution without additional reducing agents is suitable for processing solution to manufacture antimicrobial fiber containing silver, and the process is easy to control.

The antimicrobial activity of the silver can further be explained by the following reaction:

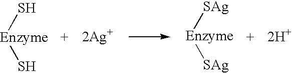

Silver nitrate is one of the most powerful chemical germicides and is widely used as a local astringent and germicide. However, the nitrates irritate the skin. Thus, it is preferable to reduce the silver nitrate to metallic silver. When the metallic silver is in contact with an oxygen metabolic enzyme of a microorganism, it becomes ionized. And, as shown in the above reaction, the silver ion interacts with the sulfhydryl group (—SH) of the enzyme in the microorganism and forms an —SAg linkage with the enzyme, which effectively blocks the enzyme activity.

The antimicrobial fiber of the present invention is prepared according to the following flow chart:

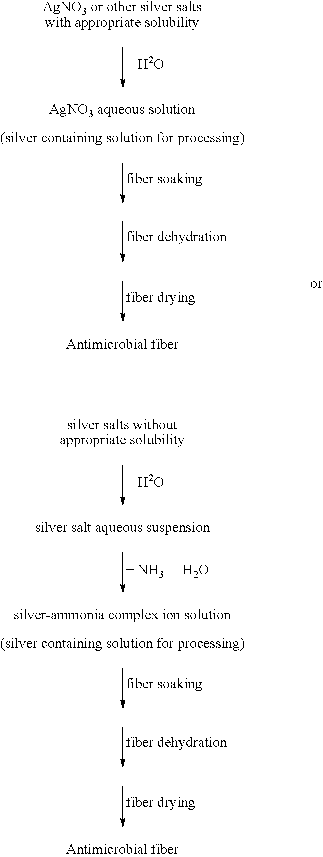

First, dissolving silver nitrate in water to form an aqueous solution of silver nitrate. Then the above solution is diluted with additional water to make the volume up to the needed. The silver containing aqueous solution is used as the soaking solution for the fiber. For 200 kg of fiber, about 1 kg-10 kg of silver nitrate, and about 500 L (liters) of water are required.

The plant fiber is preferred to be de-greased prior to the soaking. The degreased process for the fiber is commonly known in the art. After soaking in the silver containing solution for an appropriate period of time, the soaked fiber is dehydrated followed by drying under heat.

The resulting antimicrobial fiber has advantages of long-lasting effect, broad spectrum antimicrobial activity, non-toxic, non-stimulating, natural, and suitable for medicinal uses. The antimicrobial activity of the fiber is stronger when in water. Because reducing agents are not used in the process for making the antimicrobial fiber, the process is more economical and easy to control. The process of the present invention is suitable for both small scale and industrial scale production.

The following examples are illustrative, and should not be viewed as limiting the scope of the present invention. Reasonable variations, such as those occur to reasonable artisan, can be made herein without departing from the scope of the present invention.

### Example 1

### Preparation of the Small Scale of Antimicrobial Yarn

### (1) Preparation of Silver Containing Solution

(a) Silver nitrate solution:

$$\frac{{AgNO}_{3} 3.9 g}{Dissolved in 150 ml of water}$$

(b) Silver-containing solution:

The silver-containing solution was prepared by diluting the silver nitrate solution with additional water to make the volume up to 250 ml.

### (2) Preparation of Antimicrobial Yarn

The antimicrobial yarn was prepared as follows:

(a) Naturally white, degreased yarn (10 g) was immersed in the silver containing solution of (1). The yarn was squeezed and rolled in the solution so that the yarn was fully absorbed with the processing solution.

(b) The silver containing solution was partly removed from the yarn by centrifugation (such as in a washing machine) and dried in an oven at 120-160° C.

(c) The dried yarn was washed with water, and dried again in the oven to obtain the antimicrobial yarn of the present invention which showed an orange color.

### Example 2

### Preparation of Industrial Scale of Antimicrobial Yarn

### (1) Preparation of Silver Containing Solution

(a) Silver nitrate solution:

$$\frac{{AgNO}_{3} 5.5 g}{Dissolved in 200 L of water}$$

The silver nitrate aqueous solution was prepared by dissolving 5.5 kg of silver nitrate in 200 L of water at room temperature in a 500-litre container.

(b) Silver containing solution:

The silver containing solution was prepared by mixing the silver nitrate solution with the additional water. Additional water was added to the mixture to make the volume up to 500 L.

(2) Preparation of Antimicrobial Yarn

The antimicrobial yarn was prepared as follows:

(a) Naturally white, degreased yarn (200 g) was immersed in the silver containing solution of (1). The yarn was squeezed and rolled in the solution so that the yarn was fully absorbed with the silver containing solution.

The silver containing solution was partly removed from the yarn by dehydration such as using centrifugation. The yarn was further dried in an oven at 120-160° C. for about 40-60 minutes.

(b) The dried yarns were washed with water, and dried again in the oven to obtain the antimicrobial yarn of the present invention which showed a yellow-orange color.

### Example 3

### Electron Microscopic Studies of the Antimicrobial Yarn

### (1) Purpose

The yarn produced by the method described in Example 1 was analyzed for the dimension and distribution of nanosilver particles attached.

### (2) Method

Five samples of the antimicrobial yarn prepared in Example 1 (supra) was examined according to the procedure described in the JY/T011-1996 transmission electron microscope manual. JEM-100CXII transmission electron microscope was used with accelerating voltage at 80 KV and resolution at 0.34 nm.

### (3) Result

Six batches of the antimicrobial yarn samples were examined and all contained nanosilver particles which were evenly distributed to the yarn. Batch No. 010110 contained about 62% of nanosilver particles that were under 10 nm in size, about 36% that were about 10 nm, in size, and about 2% that were 15 mn in size. Batch No. 001226 contained about 46% of nanosilver particles that were under 10 nm in size, about 47% that were about 10 nm in size, and about 7% that were about 15 nm in size. Batch number 001230 contained about 65% of nanosilver particles that were under 10 nm in size, about 24% that were about 10 nm in size, and about 11% that were about 15 nm in size. Batch No. 010322-1 contained about 89% of nanosilver particles that were under 10 nm in size, about 8% that were about 10 nm in size, and about 3% that were about 15 nm in size. Batch No. 011323 contained about 90% of nanosilver particles that were under 10 nm in size, about 7% that were about 10 nm in size, and about 3% that were about 15 nm in size. Batch No. 010322-2 contained 70% of nanosilver particles that were under 10 nm in size, about 12% that were about 10 nm in size, and about 13% that were about 15 nm size. Chemical testing indicated that the silver content in the yarn was about 0.4%-0.9% by weight.

(4) Conclusion

The foregoing results demonstrated that the antimicrobial yarn contained nanosilver particles with diameters below 20 nm. These nanosilverparticles were evenly distributed to the yarn.

### Example 4

### Broad Spectrum of Antimicrobial Activity of the Yarn

### (1) Purpose

The antimicrobial yarn prepared in Example 1 was examined to determine the antimicrobial activity of the yarn.

### (2) Method

Both the antimicrobial yarn of the present invention (the experimental group) and the yarn without the attachment of nanosilver particles (the control group) were tested in the test tubes.

Microbial strains tested were Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C. These strains were either isolated from clinical cases or purchased as standard strains from Chinese Biological Products Testing and Standardizing Institute.

Two sets of test tubes, each containing a triplicate of various microbial strains were prepared by inoculating the microbial strains into the test tubes containing a meat broth. Then, equal weights of the yarns from the present invention and from the control group were inserted into the test tubes. The test tubes were then cultured at 37° C. for 18-24 hours. At the end of the incubation, an aliquot of the broth from each of the test tube was taken out and spread onto a Trypticase soy blood agar plate. The blood agar plate was incubated at 37° C. for 18-24 hours.

### (3) Results

No colony or sign of any microbial growth was observed on the blood agar plate of the experimental group, as opposed to those of the control group where signs of microbial growth were seen.

### (4) Conclusion

The antimicrobial yarn of the present invention demonstrated effective antimicrobial activity against various bacteria, fungi, and chlamydia.

### Example 5

### Long Lasting Effect of Antimicrobial Activity of the Yarn

### (1) Purpose

The antimicrobial yarn of Example 1 of the present invention was examined for the antimicrobial activity over a prolonged period of time. The antimicrobial activity of the yarn after repeated washes was also conducted.

### (2) Method

The antimicrobial yarn of the present invention was washed according to the washing procedure as provided in the Function Treatment of the Fabric, Chinese Textile Publishing House (January 2001) as follows:

(a) 2 g of neutral soap solution (1:30) was dissolved in one litre of water to obtain a wash fluid;

(b) a yarn from the experimental group or the control group as described in Example 4 was washed using the wash fluid of (a) at room temperature for 2 minutes;

(c) The yarn was rinsed in water;

(d) After every five washes in the wash fluid, the yarn was dried at 60° C.

(e) After 100 times of washing procedure according to (a) to (d), nine batches of antimicrobial yarn were tested for antimicrobial activity of Staphylococcus aureus, Escherichia coli, Candida albicans, and Pseudomonas aeruginosa according to the method provided in Example 4.

### (3) Result

No colony or any signs of microbial growth were observed in the yarn of the experimental group, as opposed to those in the control group where signs of microbial growth were observed.

### (4) Conclusion

The above results indicate that the yarn of the present invention was very effective and long lasting as antimicrobial agent even after repeated washes.

### Example 6

### Antimicrobial Activity of the Yarn Made with Different Materials or Dyed with Different Colors

### (1) Purpose

The antimicrobial activity of the yarn of the present invention prepared from different materials or dyed with various colors was examined.

### (2) Method

(a) The yarn (from the experimental group or the control group) which was made from cotton, linen, blending fabric, or which was dyed in black, blue, red, orange, and yellow was prepared.

(b) The yarns of (i) were tested for antimicrobial activity on Staphylococcus aureus, Escherichia coli, Candida albicans, and Pseudomonas aeruginosa, according to the method provided in Example 4.

### (3) Result

No colony or any signs of microbial growth were observed in the yarn of the experimental group, as opposed to those in the control group where signs of microbial growth were observed.

### (4) Conclusion

The antimicrobial yarn of the present invention made from different materials, which included cotton, linen, silk, wool, leather, blending fabric, or synthetic fiber, or dyed with different colors, was very effective as antimicrobial agent, suggesting the materials or dying methods would not and did not hinder the antimicrobial activity of the nanosilver particles-containing yarn.

### Example 7

### Preparation of Antimicrobial Nonwoven Fabric

### (1) Preparation of Silver-Containing Solution

107 g of powdered silver oxide and 100 g of citric acid hydrate was, in sequence, added to 15 L of deionized water with stirring at room temperature, forming a suspension of salt of citric acid. Concentrated ammonia water was then added to the suspension with stirring until clear solution formed. Additional water was added to the solution to make the volume up to 20 L.

### (2) Preparation of Antimicrobial Nonwoven Fabric

1 kg of nonwoven fabric was immersed in silver-containing solution to absorb the solution. The part of the absorbed solution was removed. The dehydrated fabric was dried in an oven at 120-160° C. for 40-60 minutes. After being washed with water, the fabric was dried again. Thus, antimicrobial nonwoven fabric was obtained.

### (3) Determination of Silver Content

(a) Method

USPXXII (1990)P1768

(b) Result

The content of silver of the batch 030115 is 0.59% by weight.

### (4) Electronmicroscopic Examination

(a) Method

The same as in Example 3.

(b) Result

The particle size of the sample of batch 030115 is smaller than 25 nm.

### (5) Antimicrobial Test

(a) Method

The Ministry of Health P. R. China.

<<Technical Standard For Evaluation Of Disinfectant>>

Ed. 3, Div. 1, Section: Shaken Flask Test Method

(b) Result

The sample 030115 fully (100%) inhibited 3 test microbes (E. coli 8099, S. aureus ATCC6538, C. albicans ATCC10231).

### Example 8

### Preparation of Antimicrobial Cotton

### (1) Preparation of Silver-Containing Solution

1.6 g of powdered silver oxide and 3.3 g of citric acid hydrate were, in sequence, added to 130 ml of deionized water with stirring at room temperature, forming a suspension of salt of citric acid. Concentrated ammonia water was then added to the suspension with stirring until clear solution formed. Additional water was added to the solution to make the volume up to 150 ml.

### (2) Preparation of Antimicrobial Cotton

10 g of degreased cotton was immersed in silver-containing solution and squeezed several times to fully absorb the solution. The cotton having absorbed the solution was centrifuged to remove part of the absorbed solution. The dehydrated cotton was dried in an oven at 120-160 C for 40-60 minutes. After being washed with water, the cotton was dried again, thus, antimicrobial cotton was obtained.

### (3) Determination of Silver Content

(a) Method

USPXXII(1990)P1768

(b) Result

The silver content of 4 batches (011113-1, 011113-2, 011115-1, 011115-2) is 1.32%, 1.82%, 1.24% and 1.58% by weight respectively.

### (4) Electronmicroscopic Examination

(a) Method

The same as in Example 3.

(b) Result

The particle size of the sample of 2 batches (011113-1, 011115-1) is smaller than 25 nm.

### (5) Antimicrobial Test

(a) Method

The Ministry of Health P.R. China.

<<Technical Standard for Evaluation of Disinfectant>>

Ed.3, Div. 1, Section 2.12.2 Inhibitory Circle Test Method.

(b) Result

| | Diameter of | |
|---|---|---|
| Test microbe | inhibitory circle | |
| S. aureus ATCC6538 | 16-17 | mm |
| E. coli 8099 | 15-18 | mm |
| C. albicans ATCC10231 | 7.5-9 | mm |

The diameter of inhibitory circle of the sample against 3 test microbes was larger than 7 mm. The sample 011130-1 significantly inhibited 3 test microbes.

While the invention has been described by way of examples and in terms of the preferred embodiments, it is to be understood that the invention is not limited to the disclosed embodiments. On the contrary, it is intended to cover various modifications as would be apparent to those skilled in the art. Therefore, the scope of the appended claims should be accorded the broadest interpretation so as to encompass all such modifications.

1. Plant fibers with antimicrobial activity comprising nanosilver particles which are attached to said plant fibers; wherein said nanosilver particles are 1-100 nm in diameter; and wherein said nanosilver particles-containing fibers contain 0.08%-1.8%, by weight of silver based on the total weight of said plant fibers.

2. The plant fibers according to claim 1, wherein said silver of said nanosilver particles is made by reducing silver ion dissociated from AgNO₃ or other silver salts with appropriate solubility, or silver ammonia complex ion made from silver salts without appropriate solubility and ammonia water without the use of additional reducing agent.

3. The fibers according to claim 1, wherein said fibers are made of at least one selected from the group consisting of cotton, linen, wood pulp, artificial fibers such as rayon, blended abovementioned fibers, and abovementioned fibers blending with synthetic fibers.

4. The fibers according to claim 1, wherein said fibers are in natural color or dyed with different colors.

5. The fibers according to claim 1, wherein said fibers inhibit growth of bacteria, fungi, or chlamydia.

6. The fibers according to claim 5, wherein said bacteria, fungi or chlamydia are at least one selected from the group consisting of Escherichia coli, Methicillin resistant Staphylococcus aureus, Chlamydia trachomatis, Providencia stuartii, Vibrio vulnificus, Pneumobacillus, Nitrate-negative bacillus, Staphylococcus aureus, Candida albicans, Bacillus cloacae, Bacillus allantoides, Morgan's bacillus (Salmonella morgani), Pseudomonas maltophila, Pseudomonas aeruginosa, Neisseria gonorrhoeae, Bacillus subtilis, Bacillus foecalis alkaligenes, Streptococcus hemolyticus B, Citrobacter, and Salmonella paratyphi C.

7. An antibacterial and antifungal cloth, wherein said antibacterial or antifungal cloth comprises said fibers according to claim 1.

8. The antibacterial or antifungal cloth according to claim 7, wherein said antibacterial cloth is used to treat patient with burn and scald-related skin infection, wound-related skin infection, dermal or mucosal bacterial or fungal infection, surgery cut infection, vaginitis, and acne-related infection.

9. The antibacterial cloth according to claim 7, wherein said cloth makes antibacterial clothes.

10. The antibacterial cloth according to claim 9, wherein said antibacterial clothes are at least one selected from the group consisting of underwears, socks, shoe cushions, shoe linings, bed sheets, pillow shams, towels, women hygiene products, laboratory coats, and medical robes.

11. A method for fabricating fibers with antimicrobial activity, comprising steps of:
(a) preparing a silver-containing solution which contains silver nitrate or another silver salt which is soluble in water, or contains a silver ammonia complex which is soluble in water;
(b) soaking plant fibers in said silver-containing solution and then dehydrating said plant fibers; and
(c) reducing Ag⁺ or [Ag(NH₃)₂]⁺ contained in said plant fibers into silver in a form of nano-silver particles by using cellulose of said plant fibers as a reducing agent so that said nano-silver particles are closely attached to said plant fibers.

12. The method according to claim 11, wherein said fibers are pre-degreased before soaking in said silver-containing solution.

13. The method according to claim 11, further comprising a step of treating said silver-containing solution absorbed fibers with heat at 120-200° C. for about 40-60 minutes, more preferably at 130-170° C. for about 40-60 minutes.

14. The method according to claim 11, wherein said nanosilver particle is size between 1 to 100 nm.

15. The method according to claim 11, wherein each litre of said silver-containing solution comprises 1 g-5 g of silver.

16. The method according to claim 11, wherein said fibers contain about 0.08% to 1.8%, more preferred 0.1% to 1.5% by weight of silver in a form of attached nanosilver particles.
